(19) United States
(12) Patent Application Publication
(10) Pub. No.: US 2011/0137064 A1
(43) Pub. Date: Jun. 9, 2011

(54) METHACRYLATE-BOUND PHOTOISOMERIZABLE CHROMOPHORE, METHODS FOR ITS SYNTHESIS

(75) Inventors: Andrew N. SHIPWAY, Jerusalem (IL); Nimer Jaber, Abu Ghosh (IL); Moshe Greenwald, Tel-Aviv (IL); Ariel Litwak, Ramat Hasharon (IL)

(73) Assignee: MemPile Inc., Wilmington, DE (US)

(21) Appl. No.: 12/960,994

(22) Filed: Dec. 6, 2010

Related U.S. Application Data
(62) Division of application No. 11/913,629, filed on Nov. 5, 2007, now abandoned.
Application No. 11/913,629 is a continuation-in-part of application No. PCT/IL2006/000536, filed on May 4, 2006.
(60) Provisional application No. 60/677,824, filed on May 5, 2005.

(30) Foreign Application Priority Data
May 4, 2006 (IL) IL2006/000536

(51) Int. Cl. C07C 253/30

(52) U.S. Cl. 558/339

(57) ABSTRACT

The invention discloses novel dicyanostilbene derivatives bound to a methacrylic moiety that can serve as an active chromophore in a 3-dimensional optical memory, processes for its synthesis and its intermediates.

This application is a divisional of application Ser. No. 11/913,629, filed Aug. 6, 2010, which is a Continuation-in-part of PCT/IL2006/000536 and claims the benefit of prior U.S. provisional patent application No. 60/677,824 filed May 5, 2005, the contents of which are hereby incorporated by reference in their entirety.

### FIELD OF THE INVENTION

This invention relates to a photoisomerizable choromophores, methods for its synthesis and of its intermediates.

### BACKGROUND OF THE INVENTION

Photochromic compounds are of interest in the production of many products, including sunglasses, novelty items, and high-tech items such as data storage media. While the photochromic species are generally used in the form of a doped dye, there are several advantages to a photochrome that is covalently attached to its matrix. For example, higher concentrations and better stability may be obtained. In three-dimensional optical storage, the medium is generally an organic material, which contains chromophores (the molecular data storage component) covalently bounded or embedded in a matrix (see WO 01/73,769 and U.S. Pat. No. 5,268,862). WO 01/73,769 in particular discloses stilbene derivatives as effective chromophores in three dimensional optical memories (WO 01/73,779). Photochromic media consists of chromophores which upon appropriate photochemical excitation, undergo or otherwise catalyze a change of state (e.g. by isomerization). Such a change permits the inscription (“writing”) of data. The matrix which may be polymeric (as described in WO 03/070,689) provides the required mechanical properties to the media, and ideally should be essentially inert and not interfere with the optical processes in a negative way. Stilbene is known to have a high 2-photon cross-section and short lived excited state (0.1 ns). These properties facilitate the writing of information on the memory and further the reading of the information from the memory. Both the cis and the trans isomers of stilbene have the high thermal stability important for the stability of the system and preventing loss of information.

However, the sensitivity of the medium to the storage and retrieval of data is nonetheless influenced by the properties of the matrix as disclosed in PCT/IL2006/000054. Any photochemical process is to some extent dependant on its microenvironment, for example through a solvatochromic effect in a simple case. In optical data storage, however, the effect of the microenvironment is even greater. For a chromophore to switch between the two forms that represent different data states, it must undergo a chemical transformation, which may require a volume of reaction to permit a reaction transition state. The volume of reaction means that the rate of reaction (and consequently the sensitivity of the media) is highly dependant on the free volume and viscosity of the microenvironment of the chromophore. Likewise, the existence of a reaction transition state suggests that the rate of reaction can be highly dependant on the chemical composition of the microenvironment because of interactions between the transition state and the microenvironment which may lower the transition state energy.

PCT/IL2006/000051 discloses an optical data media having a high concentration of active chromophore. Such a media having a high concentration of the active chromophore facilitates and enhances the writing of data.

### SUMMARY OF THE INVENTION

The present invention is based on the fact that a unique photoisomerizable chromophore may be synthesized and covalently linked to a methacrylate group through an alkyl spacer. Such a monomer may further be copolymerized with (alkyl)acrylates or other monomers such as styrene or maleimides to give a polymer matrix containing pendant photoisomerizable groups.

Thus, the present invention is directed to a compound of formula (I) constructed of a photoisomerizable compound linked to a methacylate:

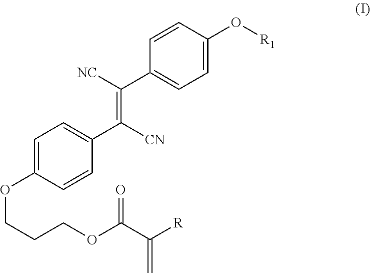

wherein R is selected from hydrogen and methyl and R₁ is selected from CH₃, CH₂CH₃, (CH₂)₂CH₃, and (CH₂)₃CH₃.

The compounds of formula (I) may either be in the cis or in the trans geometry.

The present invention is further directed to processes for the synthesis of a compound of formula (I). In a first embodiment, there is provided a commercial scale process for preparing the compound of formula (I), comprising:
a) reacting 4-hydroxybenzaldehyde with dihydropyran to yield the respective 4-tetrahydropyran (THP)-benzaldehyde of formula (A):

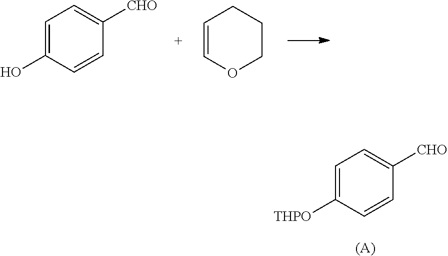

b) reacting the 4-tetrahydropyran-benzaldehyde (A) with 4-alkoxyphenylacetonitrile in the presence of a base to yield the mononitrile of formula (B):

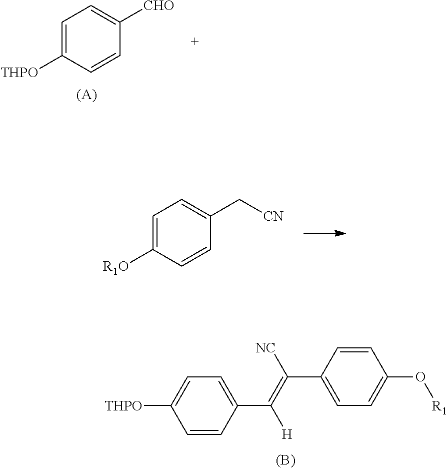

c) reacting the mononitrile of formula (B) with sodium cyanide in dimethylformamide to yield the dinitrile of formula (C):

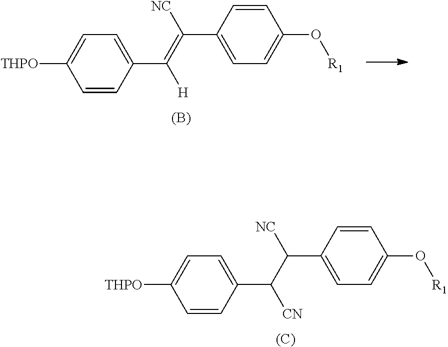

d) treating the dinitrile of formula (C) with a mild base in the presence of copper(II) acetate hydrate to afford the compound of formula (D) in a mixture of cis and trans forms;
e) deprotecting (D) to yield the compound of formula (E) as a mixture of cis and trans forms;
f) enriching the trans isomer by heating the mixture of cis and trans forms of (E) in the presence of dichlorobenzene to yield the predominantly trans-(F) compound:

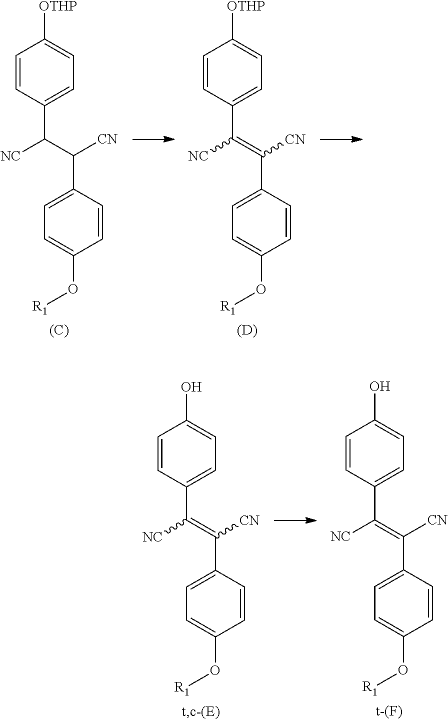

g) reacting the t-(F) with 1-chloro3-bromopropane in the presence of a base to yield the compound of formula (G):

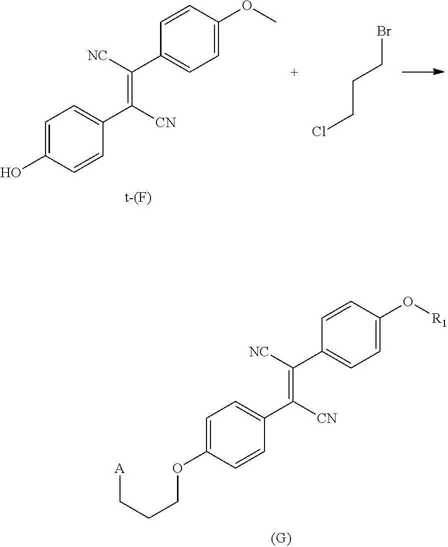

wherein A is selected from a chlorine and a bromine atom;
h) reacting the compound of formula (G) with acrylic (R═H)/methacrylic (R═CH₃) acid in the presence of a base to yield the compound of formula (I):

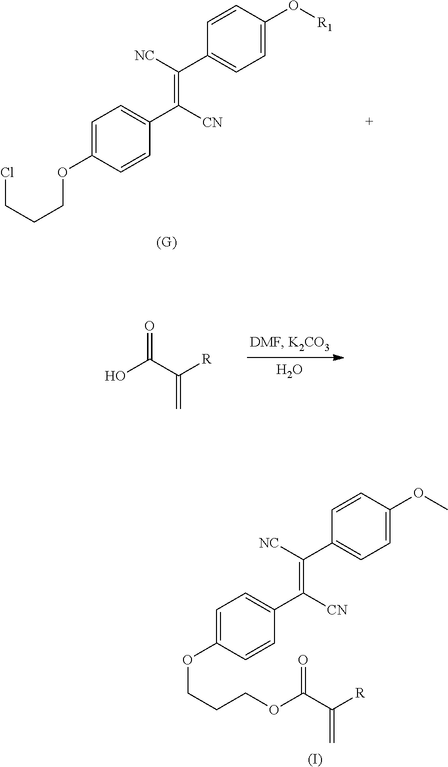

wherein R and R₁ are as defined above.

In a second embodiment, the process comprises

a) homocoupling two 4-methoxyphenyl acetonitrile molecules in the presence of I₂, NaOMe/MeOH at a low temperature to afford 4,4′-dimethoxy-α,α-dicyanostilbene of formula (II);

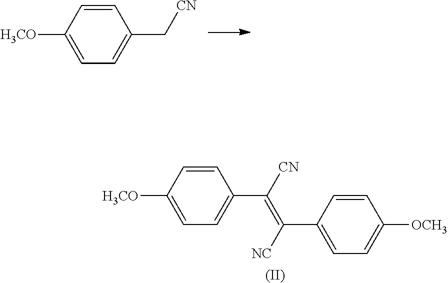

b) demethylating the compound of formula (II) by heating compound (II) in the presence of AlCl₃/pyridine and DCB to yield 4,4′-dihydroxy-α,α-dicyanostilbene of formula (III);

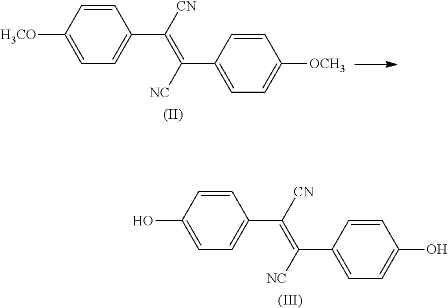

c) monoalkylating the compound of formula (III) with R₁X (wherein X is a leaving group, as known to a person skilled in the art, said leaving group being removable under such conditions known in the art, e.g., a halide selected from Br, I and Cl) in the presence of a base in acetone to yield a 4-alkoxy-4′-hydroxy-α,α-dicyanostilbene of formula (IV);

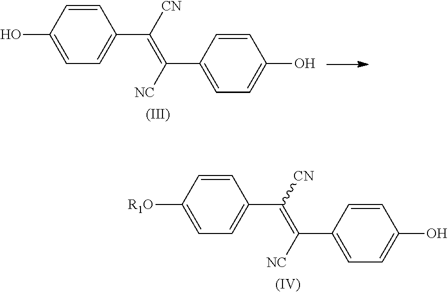

d) reacting the compound of formula (IV) with 3-bromopropyl acrylate or methacrylate in the presence of base and acetone to yield a compound of formula (I):

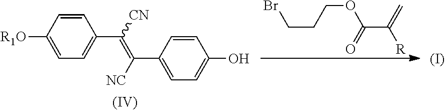

Still yet, the intermediate 2,3-bis-(4-hydroxy-phenyl)-but-2-enedinitrile (III) may by synthesized in one of the following three schemes:

i)

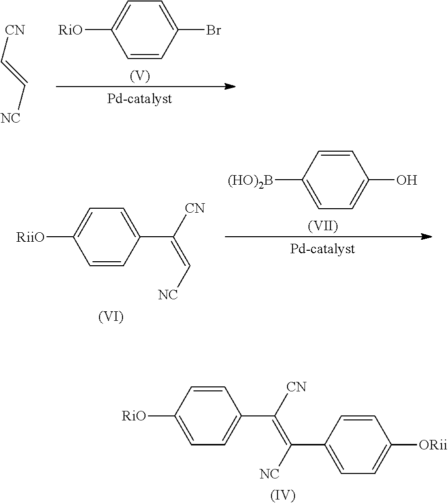

wherein Ri and Rii may be the same or different group, where one of the Ri or Rii is H, the compound is of the general formula (IV); where both Ri and Rii are H, the compound is of the general formula (III);
ii)

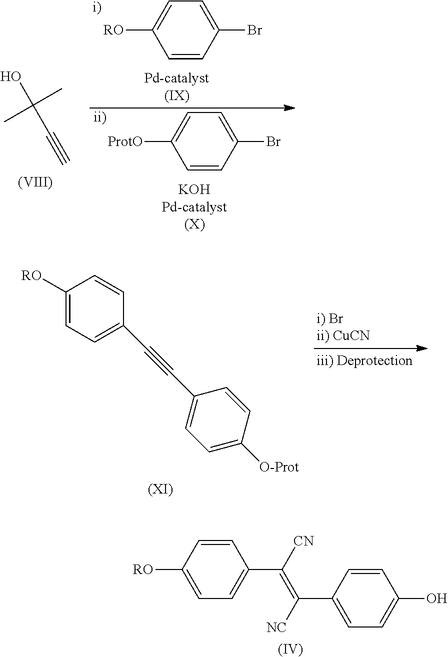

wherein “Prot” designates a protecting group which may be removed employing any one deprotection method as known to a person skilled in the art;
(iii)

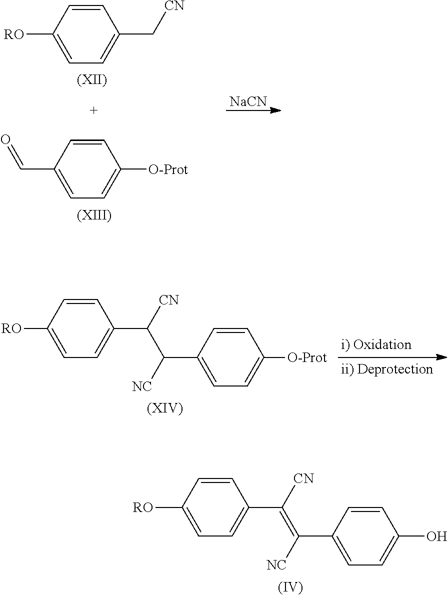

The invention is yet further directed to a three dimensional optical memory comprised of a compound of formula (I) copolymerized with other monomers.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the effect of Ultra violet irradiation of DMSDC at 254 nm. The transformation from the trans isomer (low absorbance at 260 nm) through the cis isomer to phenanthrene (high absorbance at 260 nm) is demonstrated.

FIG. 2 shows the UV-visible spectrum (changes upon irradiation) of MSDC at 460 nm irradiation. At such wavelengths the chromophore does not absorb, therefore the observed photochemistry is a result of non-linear processes.

FIG. 3 shows the ultra violet spectrum of three compounds of the α,α-dicyanostilbene in ethanol.

FIG. 4 shows the effect of deprotonation of 4,4′-dihydroxy-α,α-dicyanostilbene resulting in a red shift of 100 nm and enhancement of the charge-transfer band.

FIG. 5 shows the relaxation of the cis-DMSDC towards trans-cis (DMSDC) equilibrium at elevated temperatures as determined by NMR.

FIG. 6 shows the Arrhenius plot for the thermal rate of approach towards isomer equilibrium of DMSDC.

FIG. 7 is schematic representation of the reaction steps leading to the photoisomerizable chromophore of the present invention, 4-ethoxy,4′-hydroxy-α,α-dicyanostilbene.

FIG. 8 shows three possible routes to the synthesis of the intermediate (II-1); The Heck approach; The Sonogashira approach; and the Condensation approach.

FIG. 9 shows a schematic representation for the synthesis of the intermediate L-X—P.

### DETAILED DESCRIPTION OF EXEMPLARY EMBODIMENTS

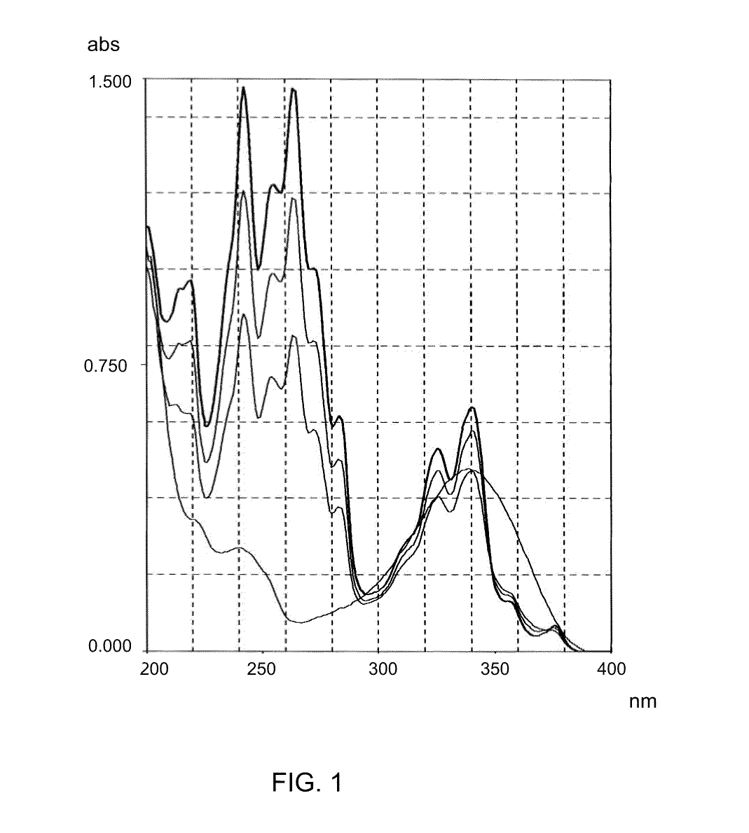

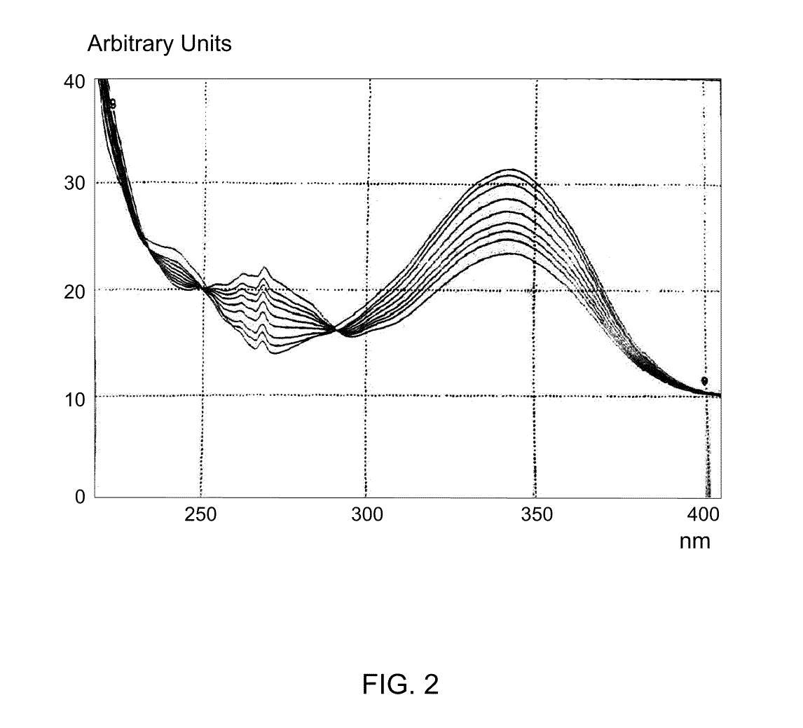

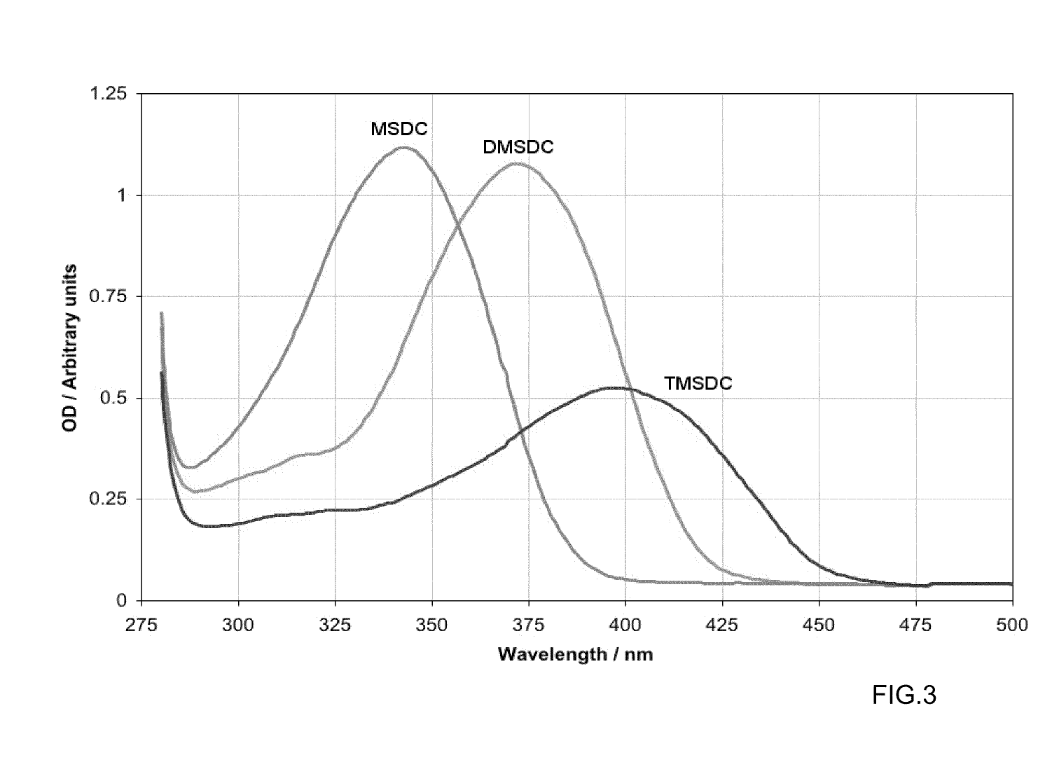

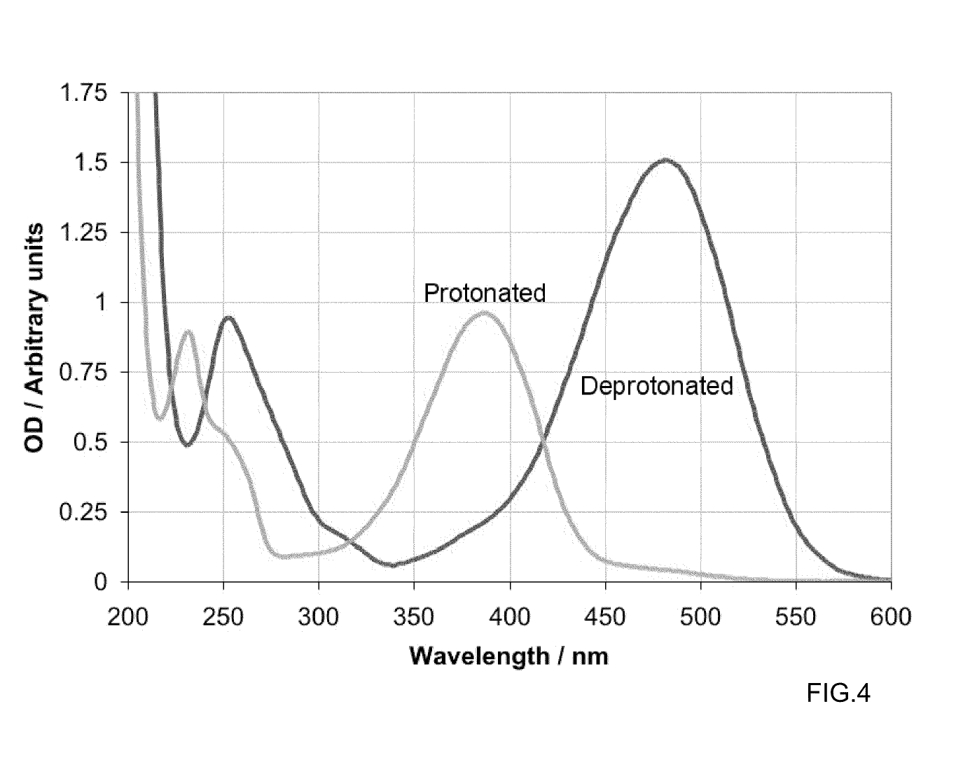

As mentioned the present invention deals with a photoisomerizable chromophore linked to an (alkyl)acrylate unit which can further be copolymerized with other monomers such as styrene or maleimides to form a polymer matrix containing the photoisomerizable chromophore where the obtained polymer serves as a 3-dimensional optical memory. An ideal chromophore, should undergo a facile, photoreaction. Various synthesized stilbene derivatives as well as stilbene itself, were examined for this key property. Under a mercury lamp (254 nm), all the examined compounds (trans-form) underwent conversion to the phenanthrene form, which requires photoisomerization to the cis-form, followed by oxidative photoisomerization to the phenanthrene. FIG. 1 demonstrates the changes in the compound trans 4,4′-dimethoxy-α,α-dicyanostilbene (DMSDC) upon irradiation. The UV spectrum demonstrates changes from the trans (low absorbance at 260 nm) isomer through the cis isomer to phenanthrene (high absorbance at 260 nm) upon irradiation at 254 nm. This process is faster for 4,4′-dimethyl-α,α-dicyanostilbene (MSDC) and much slower for carboxylic acids (data not shown), presumably because they form stable intramolecular dimers in the trans-form. As such a chemical transformation is not desirable, a different photochemistry was sought and consequently, the photoisomerization by irradiation with laser light at 460 nm was done. The chromophores do not absorb at this wavelength, so any photochemistry is the result of nonlinear processes. It was found that the formation of phenanthrenes could be avoided entirely in case where non-chlorinated solvents were used. Nevertheless, most of the examined chromophores isomerized extremely slowly or not at all. Only stilbene derivatives having nitriles connected directly to the central double bond such as MSDC could be converted at a significant rate (FIG. 2). In order to find the optimal chromophore from the family of α,α-dicyanostilbene three compounds designated MSDC (4,4′-dimethyl-α,α-dicyanostilbene), DMSDC (4,4′-dimethoxy-α,α-dicyanostilbene) and TMSDC (3,4,3′,4′-tetramethoxy-α,α-dicyanostilbene) were further studied. Analyzing the MSDC chromophore, reveals that it possesses a higher (than DMSDC) energy barrier for isomerization, and requires shorter ‘reading’ and ‘writing’ wavelengths which would require more expensive, less available laser diodes. Turning to the TMSDC, it possesses a lower (than DMSDC) energy bather for isomerization. The consequences are: significantly lower thermal stability which leads to a shorter storage time on the disc since the stored information will survive only a fraction of the time it could survive on a DMSDC based disc. TMSDC is more sensitive to elevated temperatures than DMSDC. It requires longer reading and writing wavelengths than DMSDC, which leads to lower data density and transfer rates in a working device. All three examined compounds display absorbance bands in the near UV-blue as demonstrated in FIG. 3. Examination of the band intensity over a wide concentration range shows adherence to Beer's law, demonstrating that intermolecular interactions such as aggregation do not take place. As the donating ability of the terminal groups increases (MSDC<DMSDC<TMSDC), this band moves to longer wavelengths. The shift of the absorbance toward longer wavelengths with increasing Donor-Acceptor-Donor (DAD) ability is shown particularly with another compound belonging to the family, the 4,4′-dihydroxy-α,α-dicyanostilbene. This compound may be deprotonated in situ to yield a donor-enhanced dianion. Deprotonation shifts the entire spectrum 100 nm towards the red (FIG. 4).

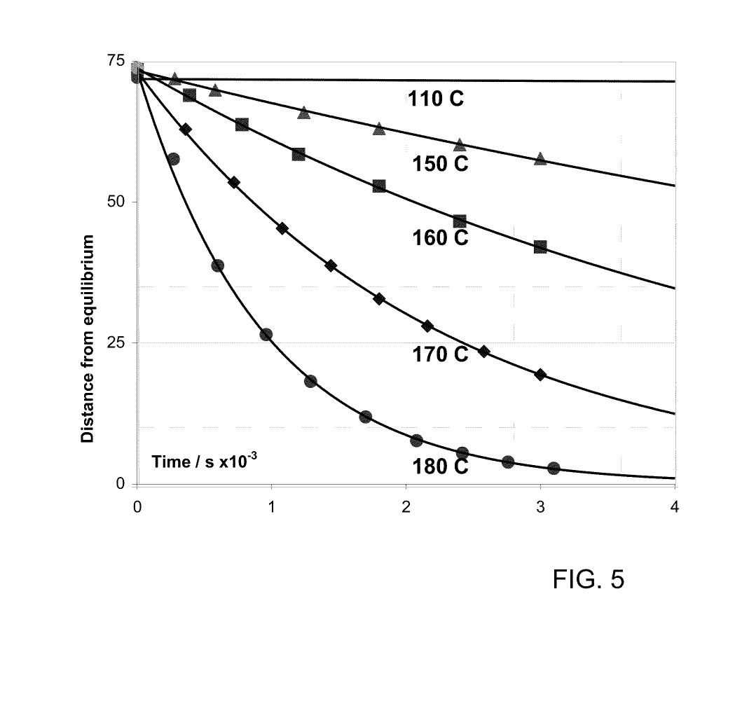

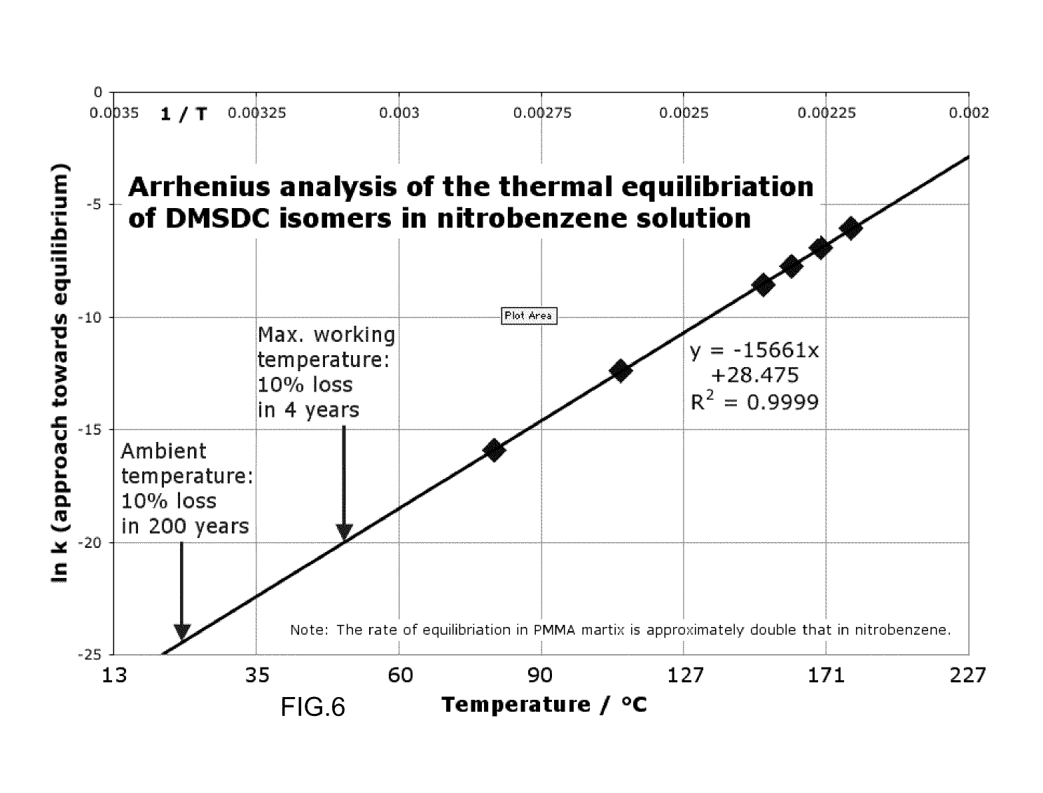

The three chromophores, MSDC, DMSDC and TMSDC were examined for their photoswitching properties. Irradiation of the trans-isomers of the chromophores with a laser at 460 nm, results in conversion to the cis-isomer to a degree of: 0% for MSDC, 18% for DMSDC and 33% for TMSDC. Similar irradiation at a lower energy of 514 nm, gave conversions of: 18% for DMSDC and 27% for TMSDC. Irradiation of the cis-isomers at 600 nm gave no conversion of MSDC and only a few percent conversion of DMSDC, but 18% conversion of TMSDC to the trans-isomer. All these results indicate that stronger DAD-architectures results in better interconversion of photoisomers (the ‘writing’ and ‘erasing’ processes in a 3D optical memory). Due to its slow isomerization, MSDC seems suitable only for a WORM-type device (one-way writing), possibly with an option to erase the entire memory simultaneously by use of heat or an intense lamp. All isomerizable systems slowly revert to equilibrium of isomers, a process that can be accelerated by raising the temperature. Qualitative studies showed that the cis-isomers are less stable than the trans-isomers (equilibria lie closer to the trans-side). The equilibrium points were measured accurately at 180° C. and, assuming ΔG to be temperature-independent, this allowed the estimation of the equilibrium point at other temperatures by use of the equation ΔG⁻-RTlnK. ΔG at 442K for DMSDC was calculated at 4.4 kJ mol⁻¹. The isomerization of cis-chromophores to equilibrium mixtures was followed by NMR at elevated temperatures in PhNO₂ (FIG. 5). The relaxation curves obtained from the NMR experiments allow the calculation for the rates of relaxation towards equilibrium, treating it as a first-order unimolecular reaction. Assuming that temperature-dependence of the rate follows an Arrhenius relationship, these data can be extrapolated to ambient and working temperatures FIG. 6 shown for DMSDC. Relaxation 10% of the way to equilibrium for MSDC, DMSDC, and TMSDC will take place in: ca. 1000, 200, and 30 years respectively at room temperature (295° K.), and ca. 15 years, 4 years, and 6 months respectively at a constant high operating temperature (325° K.). These figures suggest that TMSDC is not suitable for long-term data storage. It should be noted that relaxation towards equilibrium is not a true chemical reaction, but a mixture of the trans-to-cis and cis-to-trans processes. The calculated rate of relaxation is equal to the rates of these two processes added together. Since the equilibrium constant is the ratio between these two rates, it is possible to separate them and use the Arrhenius equation to calculate the ground state activation energies of the isomerization processes. For DMSDC, these are found to be ca. 92 kJ mol⁻¹ and 88 kJ mol⁻¹ for the trans-to-cis and the cis-to-trans processes, respectively. These figures compare with a cis-trans Ea of 154 kJ mol⁻¹ for stilbene. The best balance between stability and photoisomerizability is found in DMSDC. MSDC is more suitable for use in truly WORM memory. TMSDC, which displays the best isomerizability in the α,α-dicyanostilbene series possesses a thermal stability that is unacceptable low. Thus the above-mentioned analysis for the three α,α-dicyanostilbene compounds shows the superiority of the 4-alkoxy(methyl, ethyl, propyl, butyl or pentyl),4′-hydroxy-α,α-dicyanostilbene compound of the present invention (DMSDC) as a preferred chromophore compared to the other two above-mentioned chromophores. Thus the 3-dimensional optical memory of the present invention comprises as the photoisomerizable compound the 4,4′-dialkoxy-α,α-dicyanostilbene, where one of the alkoxy groups is either methyl or ethyl, and the other is the connection to a polymerizable group such as acrylate or methacrylate which may be polymerizable with acrylate or methacrylate to from the respective polymer having pendant 4′-alkoxy-α,α-dicyanostilbene. The resulting 3-dimensional optical memory is a polymer synthesized by copolymerization of at least two monomers. The photoisomerizable compound linked through a spacer of (CH₂)₃ to a methacrylate group forming the first monomer 4-alkoxy(methyl or ethyl)-4′-(3-(methacryloyl)propyloxy)-α,α-dicyanostilbene. The other monomer may be e.g. methyl methacrylate. Their copolymerization yields the desired 3-dimensional optical memory.

Other analogous chromophores have also been found to be less active than (I). Experiments have shown that the 2-photon isomerization of analogous compounds where the etheric substituents are replaced by esters, carbamates, and sulfonate esters proceeds significantly slower. Stilbene derivatives with amine substituents are able to isomerizes well, but they have been found to be thermodynamically unstable and have optical absorbance bands that partly obscure their fluorescence.

A commercial process for the synthesis of a compound of formula (I) comprises 6 consecutive steps as depicted in Scheme A, where the product of each step may be purified.

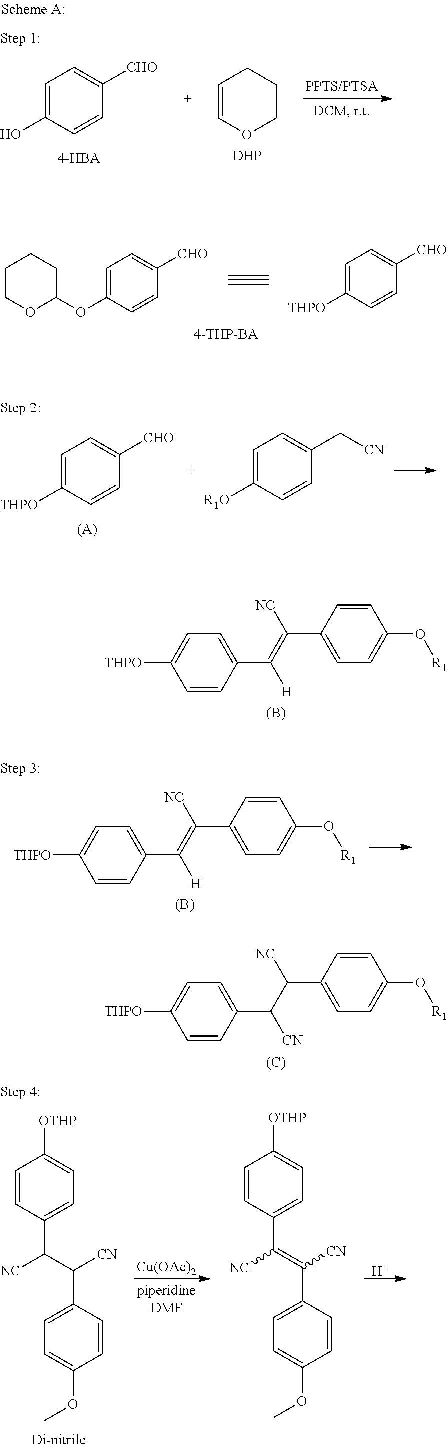

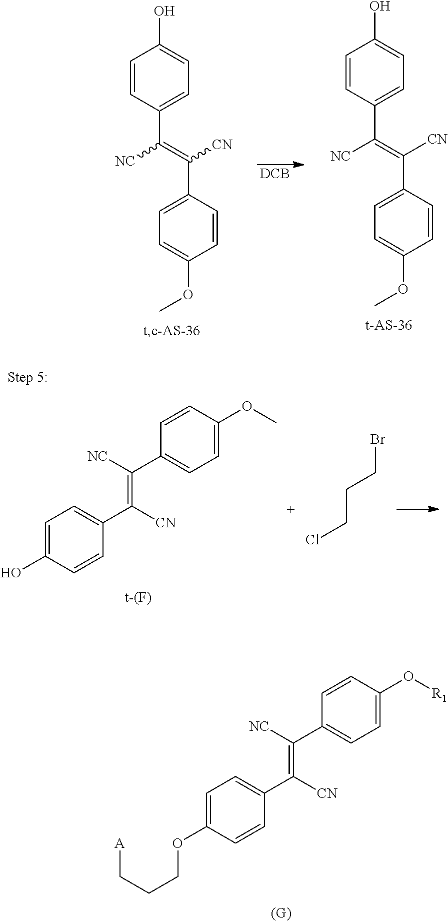

where, for example A is Cl:

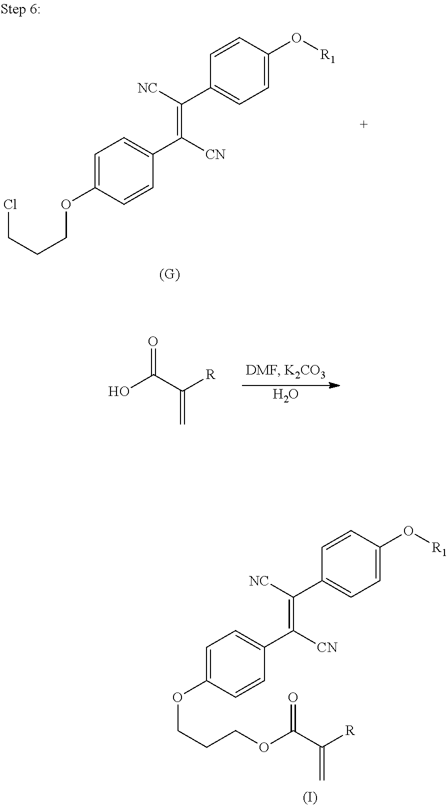

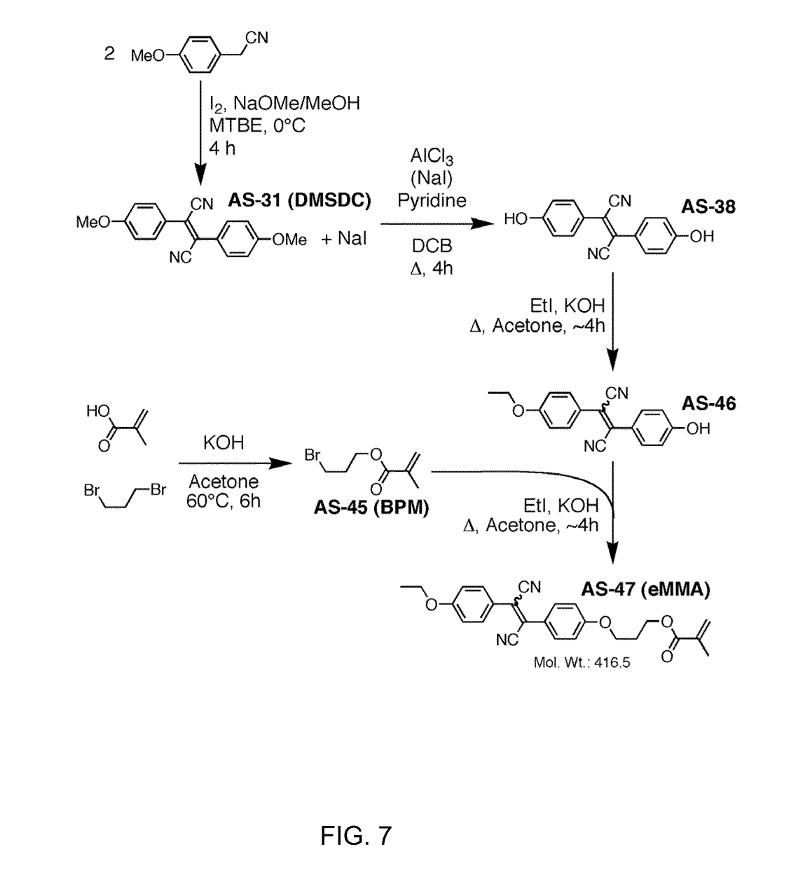

Still another procedure for the synthesis of the compound of formula (I) is done in the following manner (FIG. 7). Homocoupling of the starting material 4-methoxyphenylacetonitrile yields predominantly the trans-4,4′-dimethoxy-α,α-dicyanostilbene (AS-31). The product undergoes demethylation to yield a 4,4′-dihydroxy-α,α-dicyanostilbene (AS-38) which is further monoalkylated to yield 4-ethoxy,4′-hydroxy-α,α-dicyanostilbene (AS-46) which is reacted with 3-bromopropyl methacrylate (AS 45) to yield the desired monomer 4-ethoxy-4′-(3-(methacryloyl)propyloxy)-α,α-dicyanostilbene (AS-47). The resulting compound 4-ethoxy-4′-(3-(methacryloxy)propyloxy)-α,α-dicyanostilbene (I; designated AS-47) is isolated as the trans isomer.

The advantages of the 4,4′-dialkoxy-α,α-dicyanostilbene, in particular the 4-ethoxy,4′-alkoxy-α,α-dicyanostilbene (where the 4′ group is linked to a methacrylate) are in the mechanical and chemical stability and the optical parameters. The alkoxy(methyl, ethyl, propyl, butyl or pentyl)α,α-dicyanostilbene is stable chemically up to a temperature of 250° C. The resulting copolymer of the alkoxy(methyl, ethyl, propyl, butyl or pentyl)α,α-dicyanostilbene linked to a methacrylate with vinyl monomers is chemically and mechanically stable in the temperature range of −70° C. to +90° C. The covalent bond between the chromophore and the acrylic polymer lowers significantly the diffusion rate compared to the case of doped (unlinked) analogs. The energy barrier (activation energy) of 90 kJ/mol between the two isomeric states ensures that thermal isomerization of the 4,4′-dialkoxy-α,α-dicyanostilbene compounds is negligible at room temperature and up to 60° C. and that the compound will remain at its isomeric state for many years (ca. 50 years). The high absorption coefficient in the sub 500 nm region ensures that isomerization will occur only very slowly in the bulk of the material due to single photon absorption of regular indoor illumination. The mechanical stability of the resulting polymer disc allows high speed disc rotation for fast data manipulation (reading and writing). Furthermore, in the stilbene system the 2-photon isomerization involves only two distinct states, cis and trans and there are no side reactions of rings closures. The resulting disc comprised of the polymer is stable to water and short-chain alcohols, weak acids and bases. The 3-carbon linker between the chromophore and the methacrylate is optimal for reasons of reactivity, solubility in other monomer for the polymerization, flexibility, and molecular weight. It should be noted that a chromophore as described in WO 03/03/070,689 having a 6-carbon spacer between the chromophoric moiety and the polymerizable monomer, already at 2.5 (wt %) does not dissolve in MMA at 60° C. (boiling point of MMA is 100° C.). A shorter link would give an unacceptably reactive and inflexible connection, while a longer link would increase the molecular weight of the monomer, meaning that it could not be used in such high molar concentrations. In addition, other lengths of linkers risk modifying the chromophore's micro environment and solubility and can lead to synthetic difficulties (the low molecular-weight materials for the propyl linker are cheap and easily handled, since they may be processed by distillation methods). Another factor is the length of the alkyl chain forming the alkoxy tail (R₁). Longer alkyl chains, e.g. propyl vs. methyl would enhance the solubility. The optical parameters of the system were the following. Writing (isomerization) was achieved by 2 photons absorption in the range of 650-670 nm—the range of commercially available DVD laser diodes. Reading (fluorescence) may be achieved by 2 photons absorption in the range of 650-670 nm the range of commercially available laser diodes for DVD recording—as well as in the range of 770-810 nm—the range of commercially available laser diodes for CD recording. The fluorescence spectrum range is between 480 to 620 nm—significantly different from the absorption and excitation wavelengths, which allows for easy differentiation between the signal and the reflected laser beams and efficient collection of the signal. A disc based on acrylic polymer has high transparency and low birefringence (e.g. relative to polycarbonate and polystyrene). In particular, writing and reading experiments with both MSDC and DMSDC were done using a pulsed commercial 660 nm (DVD) laser diode radiation focused to a sub micron spot by a commercial DVD lens. For the DMSDC system, the reading fluorescence signal (2 photon absorption of 660 nm (DVD) or 780 nm (CD) laser diode radiation) was significantly altered due to selective writing. The degree of fluorescence signal alteration (modulation depth) due to prior writing depends on the laser pulse rate, pulse width and power, laser beam quality, focusing optics and laser driver parameters etc. as well as media parameters (chromophore concentration, optical quality, matrix etc.) and the signal collection system. Modulation depths of up to 10% were achieved with writing times of 2-3 seconds. Contrary to DMSDC, when MSDC was used, the reading fluorescence signal was not altered due to selective writing even in cases where the irradiation by the writing wavelength at same optical setup last more than 100s. Thus this demonstrates the superiority by means of writing efficiency of the DMSDC using commercial DVD laser diodes and commercial DVD lens over that of MSDC (ca. 30 times higher). Two photon absorption ensures high spatial selectivity of the fluorescence (reading) and isomerization (writing) processes with low cross-talk from adjacent sample volume units. This is necessary for high density data writing and reading.

It should be understood that the production process for obtaining the 3-dimensional optical memory of the present invention is not limited by the polymerization process. The resulting matrix of acrylic polymer of the present invention may be produced either by the casting method or by pressure molding methods. The former method of production, the casting method is the method of choice for small to medium volumes of production, whereas injection molding or compression molding may be used for large volumes of production.

As described above, the compound (I), wherein, for example, the tail group R₁ is ethyl, known as “eMMA” has been found to be extremely promising for use in next generation data storage media. The chromophore at the heart of this molecule is extraordinary because of its large two-photon absorption cross-section, its efficient and thermally irreversible photochromism, its large stokes shift, and several other properties.

However, in order to successfully commercialize products incorporating (I), its manufacture must be available in multi-ton quantities and at a low cost. As mentioned above, the present invention concerns an improved 6-step method for the synthesis of the compound of formula (I). The present invention however concerns another approach serving as an alternative to the previously described methods (WO 03/070,689) for the synthesis of (I) which are actually prove to be a low yield procedure, expensive and environmentally damaging reagents, and unscalable procedures. For example, the synthesis depicted in FIG. 7 relies on the use of large amounts of molecular iodine as an oxidizing agent, introduces the asymmetry in the molecule by a statistical approach, utilizes silica gel for purifications, and requires the distillation of temperature-sensitive materials—a process that can not scaled to large processes.

Hence the present invention concerns an improvement by synthesizing compound (I) from the key intermediate 2,3-bis-(4-hydroxy-phenyl)-but-2-enedinitrile (prepared as demonstrated above or a derivative thereof such as AS-46 of FIG. 7) and L-X—P, where L is a leaving group and in particular bromide, X is a (CH₂)₃ and P is a polymerizable group, in particular an acrylic or methacrylic moiety which are reacted together under Williamson conditions to give the final product. The yields (Example 1) are high.

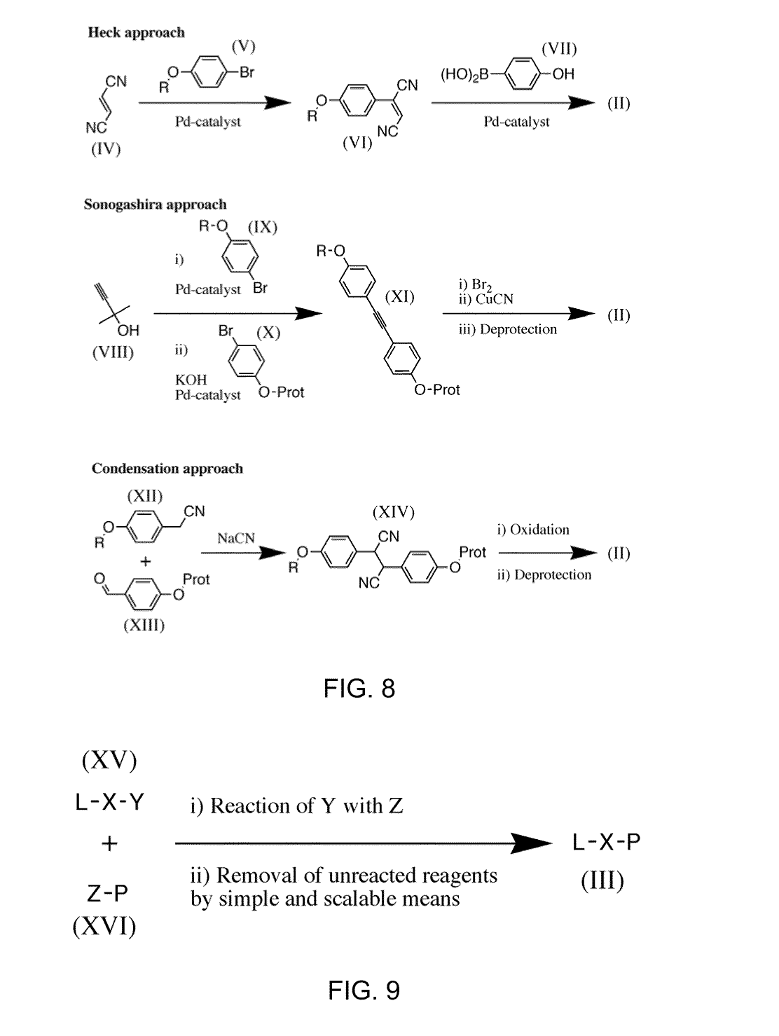

Three synthetic approaches to synthesize the key intermediate 2,3-bis-(4-hydroxy-phenyl)-but-2-enedinitrile are given in FIG. 8. The first approach is a stepwise Heck reaction, where two different aromatic molecules (V and VII) are coupled in turn to a fumaronitrile core. The second is based on a one-pot tandem Sonogashira coupling of halides (IX) and (X) to the protected acetylene (VIII), which results in a diaryl acetylene (XI), which is further brominated and then substituted with nitriles. The third is a condensation of a protected benzaldehyde (XIII) with a phenyl acetonitrile (XII) in the presence of cyanide, which leads to a dicyanodiarylethane (XIV), which is in turn oxidized to give the central double bond. All of these reaction pathways result in the product (IV). The second and third approaches require the use of protecting group chemistry (Prot=protecting group), where the protecting group may be any protecting group that is stable under the critical reaction conditions. The first and second approaches utilize palladium-catalyzed cross-coupling chemistry, which can be realized on a large scale by the recycling of the catalyst.

The resulting key intermediate, 2,3-bis-(4-hydroxy-phenyl)-but-2-enedinitrile or a derivative thereof such as AS-46 shown in FIG. 7 is then reacted with the L-X—P. The L-X—P entity is prepared according to the general scheme depicted in FIG. 9. The groups Y and Z react together to couple the two parts of the product. They are chosen not only so that the reaction is economical and facile, but also such that excess unreacted reagents can be easily removed at the end of the reaction by scalable methods. Examples of suitable L-X—Y reagents include 3-bromopropanol (which can be removed by an aqueous wash) and 1-chloro-3-bromopropane (which can be removed by distillation under reduced pressure).

Experimental

### EXAMPLE 1

The photoisomerizable compound (AS-31 in FIG. 7) (4,4′-dimethoxy-alpha,alpha-dicyanostilbene) was synthesized by the action of molecular iodine and sodium methoxide on (4-methoxyphenyl)acetonitrile. Sodium metal (17 g) was dissolved in MeOH (150 mL), and the resulting solution was added over 2 hours to a stirring solution of (4-methoxyphenyl)acetonitrile (50 mL, 0.37 mmol), THF (250 mL) and I₂ (93 g) at −5° C., under an inert atmosphere. The yellow mixture was then stirred a further 15 minutes, after which the solvents were removed under vacuum. The resulting solid was partitioned between dichloromethane (500 mL) and 0.025 M sodium thiosulfate (400 mL). The organic layer was collected, combined with 2 extractions (100 mL) of the aqueous layer, dried over magnesium sulfate, filtered, and finally condensed to ca. 50-100 mL. The yellow crystals were filtered off and washed with ether, giving pure trans-(1) (20.5 g, 38%). The remaining solution was condensed, and MeOH (100 mL) was added. More crystals formed, which were collected giving (1) (10.8 g, 20%). The remaining solution was condensed and chromatographed to give additional cis-(1) (14.5 g, 27%). Total Yield=86%. Analyses for the trans-isomer: ¹H NMR: m, 7.79; m, 7.01; s, 3.88. ¹³C NMR: 162.0; 130.4; 124.6; 122.7; 117.3; 114.6; 55.5. EA: Expected (C 74.47, H 4.86, N 9.65), Rcvd (C 74.27, H 4.83, N 9.61).

The photoisomerizable chromophore (AS-31 in FIG. 7) was then demethylated by the action of aluminium chloride in the presence of pyridine and sodium iodide to give (AS-38—FIG. 7) (4,4′-dihydroxy-alpha,alpha-dicyanostilbene). trans-AS-31 (20.0 g, 35 mmol) and NaI (20 g) were suspended in toluene (500 mL) under an inert atmosphere, then pyridine (20 mL) and AlCl₃ (20 g) were added. The reaction was protected from light and stirred at reflux for 2 days. The finished reaction was decomposed with 10% HCl (200 mL) while hot, was cooled, then the crude product was collected by filtration and recrystallized from MeCN. Pure trans-(2) is obtained (17.2 g, 96%). ¹H NMR: s, 9.21; m, 7.74; m, 7.02. ¹³C NMR: 161.0; 131.4; 124.9; 123.6; 118.1; 116.8. EA: Expected (C 73.27, H 3.84, N 10.68), Rcvd (C 73.36, H 3.99, N 10.94).

The diphenol (AS-38 in FIG. 7) was monoalkylated by ethyl iodide to give the phenol (AS-46 in FIG. 7) (4-ethoxy,4′-hydroxy-alpha,alpha-dicyanostilbene). (AS-38) (30.0 g) and KOH (7.0 g) were dissolved in acetone (150 mL) under an inert atmosphere. The mixture was brought to reflux, iodoethane (15 mL) was added, and reflux was continued for 3 h, by which time the red reaction mixture had turned orange. The mixture was cooled, sufficient HCl was added to obtain a yellow color, and most of the solvent was removed. The mixture was then taken up in DCM (150 mL), filtered, and the solid was washed with DCM. [The solid was washed with water and dried to give recovered starting material (38%)]. The DCM solution was evaporated to dryness, then was taken up in 0.5 M NaOH (200 mL). The resulting suspension was filtered and the solid was washed well with water. [The solid is the bis-ethylated product (21%)]. Conc. HCl was added to the basic solution until a yellow color was obtained, then the precipitate was collected by filtration, washed with water, and dried to give (AS-46) as a yellow solid (32%). ¹H-NMR (CDCl₃, 298 K, 300 MHz, trans-isomer): m, 7.7-7.8; m, 6.9-7.0; q, 4.1; t, 1.45. ¹³C-NMR (CDCl₃, 298 K, trans-isomer): 162.3; 161.2; 131.5; 131.3; 116.9; 115.8; 64.6; 14.9. EA: Expected for 46·0.5 H₂O (C 72.23, H 5.05, N 9.36), Revd (C 72.53, H 4.99, N 9.29).

Finally, the phenol (AS-46 in FIG. 7) was alkylated with 3-bromopropyl methacrylate (L-X—P in FIG. 7) to give the final product (AS-47) (4-ethoxy-4′-(3-(methacryloyloxy)propyloxy)-alpha,alpha-dicyanostilbene). A trans-cis mixture (ca. 15% cis) of (3) (5.0 g, 17 mmol) was dissolved in dry MeCN (40 mL) under nitrogen, then K₂CO₃ (7.5 g, 54 mmol) and 3-bromopropyl methacrylate (5 g, 24 mmol) were added. The mixture was stirred at reflux for 18 hours after which it had become yellow-orange. The reaction mixture was cooled and filtered, the solid was washed with DCM, then the solvents were removed under vacuum. The resulting thick liquid was dissolved in ether (50 mL) and left in the fridge overnight to crystallize. The crystals were then collected by filtration and dissolved in chloroform. The solvents were removed and the thick liquid remaining was taken up in MeOH (50 mL) and left in the fridge to crystallize. The crystals were collected and washed with a little MeOH. They were found to be pure trans-(AS-47) (4.4 g). The supernatants from the crystallizations were combined, condensed, and subjected to chromatography (2:1 hexane:chloroform) to give additional trans-(AS-47) (0.24 g) and a 4:1 cis: trans-(4) mixture (1.90 g). In all, 47 was obtained in a 91% yield. ¹H-NMR (CDCl₃, 298 K, 300 MHz, assignments are unconfirmed): Trans-isomer—m, 7.88 (2H, Ar); m, 7.00 (2H, Ar); m, 6.12 (1H, C═CH₂) m, 5.58; (1H, C═CH₂); t, 4.36 (2H, OCH₂); m, 4.0-4.2 (4H, OCH₂); m, 2.2 (2H, CH₂); m, 1.95 (3H, CH₃C═CH₂); t, 1.46 (3H, CH₂CH₃). Cis-isomer—m, 7.27 (2H, Ar); m, 6.80 (2H, Ar); m, 6.12 (1H, C═CH₂) m, 5.58; (1H, C═CH₂); t, 4.33 (2H, OCH₂); m, 4.0-4.2 (4H, OCH₂); m, 2.2 (2H, CH₂); m, 1.94 (3H, CH₃C═CH₂); t, 1.44 (3H, CH₂CH₃). ¹³C-NMR (CDCl₃, 298 K): Trans-isomer—167.3; 161.5; 161.1; 138.2; 130.5; 130.4; 125.6; 124.8; 124.4; 122.9; 122.4; 117.3; 117.3; 115.0; 115.0; 64.8; 63.8; 61.2; 28.5; 18.3; 14.6. EA: Expected (C 72.10, H 5.81, N 6.73), Revd (C 71.90, H 5.59, N 6.71).

The following examples exemplify the reaction conditions and workups for the preparation of the intermediates (II) and (III) that have been used for specific embodiments of the reactions contained in the general reaction schemes (FIGS. 8 and 9), giving the desired reactions.

### EXAMPLE 2

### Heck Approach (R=Et)

Fumaronitrile (2.24 mmol) is added to a mixture of 4.27 mmol 4-bromo-phenetole, potassium acetate (5.6 mmol), tetrabutylammoniumbromide (2.45 mmol), palladium acetate (0.11 mmol) and DMF, and is kept under nitrogen atmosphere at room temperature. The mixture is heated at 80° C. under magnetic stirring for 3 days, then cooled to room temperature. Water is added and the mixture was partitioned with diethyl ether. The organic layer is washed with brine and water, dried with sodium sulfate, and the solvent is evaporated. The residue is the crude 2-(4-ethoxy-phenyl)-but-2-enedinitrile, which may be expected to give ˜77% yield after purification. {Procedure based on: M. Moreno-Manas, R. Pleixats, A. Roglans, Synltt. 1997, 1157}.

The 2-(4-ethoxy-phenyl)-but-2-enedinitrile obtained in Example 2 is added to a dry flask with a nitrogen inlet (0.2 mol), together with 5 mol % Pd(OAc)₂, 0.4 mol Cu(OAc)₂ and DMF, The mixture is stirred for 0.5 h, then 0.24 mol 4-hydroxyphenylboronic acid and 0.6 mol of LiOAc is added and the reaction mixture is heated to 100° C. for 3 h. Water is added and the organic phase is extracted with chloroform. The organic layer is washed with brine and water, dried with MgSO₄, and the solvent is evaporated. The residue is the crude product, which may be expected to give ˜86% yield after purification. {Procedure based on: Du X, Suguro M, Hirabayashi K, Mori A, Nishikata T, Hagiwara N, Kawata K, Okeda T, Wang H F, Fugami K, Kosugi M., Org Lett. 2001, 3(21), 3313}.

### EXAMPLE 3

### Example Sonogashira Approach (R=Et)

Protection: A solution of 1.0 mmol of 4-bromophenol in 10 mL dichloromethane is stirred at room temperature under nitrogen and 0.9 mol % of pyridinium p-toluenesulfonate is added, followed by the dropwise addition of dihydropyran (1.2 mmol). The mixture is stirred at ambient temperature for 8 h and then diluted with 30 mL ether. The organic layer is washed with two portion of brine and is then dried with anhydrous MgSO₄. The solvent is removed under vacuum to give crude 2-(4-Bromo-phenoxy)-tetrahydropyran, which gives an expected yield of ˜93% after purification. {Procedure based on: J. Luis, J. Augusto R. Rodrigues, J. Braz. Chem. Soc., 2003, 14(6), 975}.

Coupling: 10 mmol 4-bromophenetole, 0.5 mmol PdCl₂(PPh₃)₂ and 0.5 mmol CuI are placed into a flame dried Schlenk flask. 20 ml diisopropylamine is added to the flask followed by 13 mmol 2-methyl-3-butyn-2-ol. The reaction mixture is stirred under argon for one hour at 50° C. Then 80 mmol KOH, 0.5 mmol PdCl₂(PPh₃)₂, 0.5 mmol CuI and 10 mmol of 2-(4-Bromo-phenoxy)-tetrahydropyran are added and the reaction mixture is heated for five hours at 110° C. After cooling to room temperature the KOH is neutralized with 1M HCl, and then the mixture is extracted with dichloromethane. The combined organic phases are dried over MgSO₄ then evaporated. The crude 2-[4-(4-Ethoxy-phenylethynyl)-phenoxy]-tetrahydro-pyran gives an expected yield of 72% after purification. {Procedure based on: Z. Novak, P. Nemes, A. Kotschy, Org. Lett. 2004, 6(26), 4917}.

Bromination: A round bottomed flask is fitted with a magnetic stirrer and dropping funnel. Glacial acedic acid, 20 mmol of 2-[4-(4-Ethoxy-phenylethynyl)-phenoxy]-tetrahydro-pyran and 100 mmol LiBr are added, then 20 mmol of bromine is added dropwise over 1 h. The reaction mixture is stirred for 6 h. Water is added and the product is extracted with chloroform. The extract is washed with 5% NaHCO₃ and then dried over MgSO₄. Evaporation of the solvent leads to 2-{4-[1,2-Dibromo-2-(4-ethoxy-phenyl)-vinyl]-phenoxy}-tetrahydro-pyran in an expected ˜83% (˜97% trans). {Procedure based on: J. Konig, V. Wolf, Tetrahydron Lett. 1970, 19, 1629}.

Cyanation: A round bottomed flask, fitted with a magnetic stirrer, a condenser and a nitrogen inlet tube, is charged with 0.5 mmol copper(I) cyanide and dry DMF. This stirred mixture is heated to reflux for 1 h and then allowed to cool to room temperature under a nitrogen atmosphere. 2-{4-[1,2-Dibromo-2-(4-ethoxy-phenyl)-vinyl]-phenoxy}-tetrahydro-pyran (0.20 mmole) is added and the stirred solution is heated in an oil bath maintained at 130° C. for 12 h. After cooling to room temperature, the cooled mixture is poured into 6M aq NH₃, the resulting blue solution is stirred for 1 h and then vaccum filtration. The precipitate is washed with Et₂O. All the ether portions are combined, washed once with water, once with brine, dried over MgSO₄, filtrated and reduced on a rotary evaporation. The pure 2-(4-Ethoxy-phenyl)-3-[4-(tetrahydro-pyran-2-yloxy)-phenyl]-but-2-enedinitrile is recrystallized from methanol to give an expected yield of ˜79%.

Deprotection: 10 mmol of 2-(4-Ethoxy-phenyl)-3-[4-(tetrahydro-pyran-2-yloxy)-phenyl]-but-2-enedinitrile in methanol and 0.9 mol % of pyridinium p-toluenesulfonate are heated at 50° C. for 6 h. Vacuum filtration leads to isolation of pure (II). {Procedure based on: J. Luis, J. Augusto R. Rodrigues, J. Braz. Chem. Soc., 2003, 14(6), 975}.

### EXAMPLE 4

### Demonstrated Condensation Approaches (R=Me)

5A: Using MOM-Protection

Protection: 4-Hydroxy-benzaldehyde (1.0 g, 8.2 mmol) was dissolved in DMF (10 mL), under N₂. K₂CO₃ (1.1 eq., 9.0 mmol, 1.25 g) was added and the mixture was stirred for 15 min at room temperature. MOM-Cl (0.70 ml, 9.0 mmol, 1.1 eq.) was added dropwise via a syringe and the mixture was stirred overnight. TLC (Hexane/EtOAc 2:1) indicated almost complete conversion. Two drops of MOM-Cl and 30 mg of K₂CO₃ were added to complete the reaction. The mixture was poured into water (80 ml) and extracted with diethylether (2x 50 mL). The combined organic extracts were washed with water (2×20 mL), brine, dried over Na₂SO₄ and evaporated under reduced pressure. The crude product (4-Methoxymethoxy-benzaldehyde, 1.2 g, 89%) was used in the next step without further purification.

¹H-NMR: CDCl₃, δ ppm=9.89 (s, 1H), 7.82 (d, 9.0 Hz), 7.13 (d, 9.0 Hz), 5.24 (s, 2H), 3.48 (s, 3H)

Coupling: MeOH (9 ml) was added to a stirred solution of NaCN (18 mmol, 870 mg) in water (3 mL) and the mixture was heated to reflux. 4-Methoxyphenylacetonitrile (5, 0.98 g, 6.7 mmol) was added in one portion. Second portion of 4-Methoxyphenylacetonitrile (5, 0.44 g, 3 mmol) was mixed with 4-Methoxymethoxy-benzaldehyde (1.2 g, 7.3 mmol), dissolved in 5 ml of MeOH and slowly added (during 40 min) to the hot reaction mixture. After the addition was complete, the mixture was stirred for an additional 40 min, then cooled to ambient and filtered. The precipitate was washed with 75% MeOH, water, again 75% MeOH, ether and dried in air. Only 350 mg (15%) of 2-(4-methoxymethoxy-phenyl)-3-(4-methoxy-phenyl)-succinonitrile was obtained. The filtrate was extracted with CH₂Cl₂ (3×50 ml), the combined organic extracts were evaporated, dissolved in water: MeOH (3 mLl:12 mL), treated with NaCN (800 mg) and refluxed overnight. Cooling to ambient and usual work-up afforded an additional 350 mg of 2-(4-methoxymethoxy-phenyl)-3-(4-methoxy-phenyl)-succinonitrile (total 30%).

¹H-NMR: CDCl₃, δ ppm=7.03 (d, 9.0 Hz), 7.02 (d, 9.0 Hz), 7.00 (d, 9.0 Hz), 6.77 (d, 9.0 Hz), 5.08 (s, 2H), 4.16 (s, 2H), 3.71 (s, 3H), 3.37 (s, 3H).

Oxidation: Crude product 2-(4-Methoxymethoxy-phenyl)-3-(4-methoxy-phenyl)-succinonitrile (300 mg, 0.93 mmol) was dissolved in DMSO (20 ml) at room temperature. Cu(OAc)₂ (30 mg) was added, followed by N-methyl-aminoethanol (700 mg, ˜10 eq.). The flask was covered with aluminium foil and the mixture was stirred overnight with air bubbling through the solution. TLC showed almost complete conversion, with absence of other by-products, and the HPLC indicated a 2:1 isomer ratio. The mixture was exposed to light, stirred for an additional 2 hr and analyzed again by HPLC, to show a ˜1:1 isomer ratio.

The mixture was poured into water (150 mL), acidified with 3M HCl (10 mL) and extracted with CH₂Cl₂ (2×50 mL). The combined organic extracts were evaporated under reduced pressure and the crude residue (2-(4-Methoxymethoxy-phenyl)-3-(4-methoxy-phenyl)-but-2-enedinitrile, 280 mg, 94%) was used in the next step without further purification.

Deprotection: Crude 2-(4-Methoxymethoxy-phenyl)-3-(4-methoxy-phenyl)-but-2-enedinitrile (280 mg, 87 mmol), obtained in example 3, was dissolved in a mixture of MeOH (20 ml), THF (3 ml) and 3M HCl (3 ml). The mixture was first stirred at ambient for 2 hr, showing almost no conversion (TLC, Hexane/EtOAc 2:1). The mixture was refluxed for 6 hrs to complete the hydrolysis. Two isomers of the desired material were detected on TLC and no by-products were observed. The mixture was diluted with water (100 ml) and extracted with CH₂Cl₂ (2×50 ml). The combined organic extracts were washed with brine, dried over MgSO₄ and evaporated under reduced pressure, affording the crude semi-solid 1 (220 mg) as a mixture of 2 isomers. Trituration with Hexane/Et₂O caused formation of a crystalline precipitate of (II), which was filtered off and analyzed by HPLC and NMR. A ˜4:1 ratio between 2 isomers was observed.

Isomer A: ¹H-NMR: CDCl₃, δ ppm=7.80 (d, 9.0 Hz), 7.75 (d, 9.0 Hz), 7.04 (d, 9.0 Hz), 6.97 (d, 9.0 Hz), 6.14 (br s, 1H), 3.91 (s, 3H). Isomer B: ¹H-NMR: CDCl₃, δ ppm=7.31 (d, 9.0 Hz), 7.27 (d, 9.0 Hz), 6.85 (d, 9.0 Hz), 6.79 (d, 9.0 Hz), 5.90 (br s, 1H), 3.85 (s, 3H).

5B: Using DHP-Protection

Protection: A solution of 3,4-dihydro-2H-pyran (5.6 ml, 61 mmol), in dichloromethane (10 ml) is added dropwise, over 3 hrs, to a stirred suspension of 4-hydroxybenzaldehyde (5.0 g, 41 mmol) and pyridinium p-toluene sulfonate (205 mg, 0.8 mmol) in dichloromethane (140 ml). The mixture is stirred at room temperature overnight, then washed with brine (3×50 ml), dried over MgSO₄ and evaporated. If purity is not sufficient, the crude product obtained may be passed over a short silica column using 20% EtOAc/P.E. as eluent. 4-(Tetrahydro-pyran-2-yloxy)-benzaldehyde obtain as yellow oil (8.0 g, 95% yield). {Procedure based on D. Pez et al, Bioorg. Med. Chem., 2003, 11, 4693-4711, and the demonstrated analogous reaction above}.

Coupling: A three-necked round bottom flask is fitted with dropping funnel and a condenser. Water (2.0 ml) and NaCN (1.23 g, 25 mmol) are added to and warming is started. When. NaCN is dissolved MeOH (8.0 ml) is added and mixture is warmed to reflux. 4-Methoxyphenylacetonitrile (1.24 g, 8.4 mmol) is added at once, followed by dropwise addition (1 hr.) of a solution of 4-(Tetrahydro-pyran-2-yloxy)-benzaldehyde (2.06 g, 10 mmol) and -Methoxyphenylacetonitrile (0.77 g, 5.2 mmol). The reaction is stirred for additional 2 hrs and cooled to room temperature. The crude product is collected by suction filtration and washed with water, aqueous methanol end finally with t-butylmethyl ether/P.E. mixture. 2.57 g of 2-(4-Methoxy-phenyl)-3-[4-(tetrahydro-pyran-2-yloxy)-phenyl]-succinonitrile (71%) were obtained. {Procedure based on R. B. Davis and J. A. Ward, Org. Synth., collective volume 4, 392-395, John Wiley & Sons, Inc, and the demonstrated analogous reaction above}.

Oxidation: A solution of 2-(4-Methoxy-phenyl)-3-[4-(tetrahydro-pyran-2-yloxy)-phenyl]-succinonitrile (0.5 g, 1.4 mmol), Cu(OAc)2 monohydrate (14 mg, 5% eq) and piperidine (0.14 ml, 1.4 mmol) in DMF (3.7 ml) is stirred overnight at room temperature with internal air bubbling. Reaction mixture is diluted with water (20 ml), pH is adjusted to ˜2, using 1M HCl. The product (2-(4-Methoxy-phenyl)-3-[4-(tetrahydro-pyran-2-yloxy)-phenyl]-but-2-enedinitrile) may be either collected by suction filtration or extracted with chloroform. Typical yield is 95%. {Procedure is based on R. B. Davis, U.S. Pat. No. 2,851,477, and the demonstrated analogous reaction above}.

Deprotection: 2-(4-Methoxy-phenyl)-3-[4-(tetrahydro-pyran-2-yloxy)-phenyl]-but-2-enedinitrile (7, 0.4 g, 1.1 mmol) and pyridinium p-toluene sulfonate (3.0 mg, 1% eq.) in methanol (4 cc) were heated at reflux for 6 hrs. Solvent is evaporated and the crude product is dissolved in chloroform (10 ml) and washed with water (3×5 ml) and brine (1×5 ml). After drying over MgSO₄ and evaporation (II) is obtained as a yellow powder (295 mg, 97%).

### EXAMPLE 5

### Demonstrated Syntheses of L-X—P

From 3-bromopropanol: A 100 ml three-necked round-bottomed flask is fitted with a magnetic stirring bar, Dean-Stark apparatus and an extended dropping funnel. The flask was charged with 40 mmol 3-bromo-1-propanol, 0.6 mmol para-toluene sulfonic acid, 0.6 mmol BHT and toluene. The flask was heated to a reflux by oil bath. A solution of 50 mmol methacrylic acid in toluene was added to the dropping funnel. The methacrylic acid solution was added dropwise slowly over a period of 3-4 h, during the dropping stage, a white solid precipitate was established. After the dropping was finished, the reaction mixture was heated for another 13 h. The reaction flask was allowed to reach to room temperature. 50 ml of petroleum ether was added to the reaction mixture and another white solid precipitate. Then the white solids were filtrated by vacuum. The white solid precipitate was introduced into oven in 55° C. for drying to lead 0.1-1.2 g of white powder.

The filtrate was transferred to a 250 ml separatory funnel and washed with 1M NaOH, then with twice of water. The organic phase was dried over granulated CaCl₂, filtrated and the solvents were removed under vacuum to give 77% of slightly yellowish liquid of the product (3-bromopropyl methacrylate). The purity of the product by NMR was 95%, while the other 5% residue of impurities is toluene (4.8%) and BHT (0.2%).

From 1-chloro-3-bromopropane: A 100 ml three-necked round-bottomed flask is fitted with a magnetic stirring bar, distillation apparatus and dropping funnel, was fitted with 50 mmol methacrylic acid, 150 mmol 1-chloro-3-bromopropane and DMF. The temperature was raised to 80° C. 60 mmol of 25% NaOCH₃ in methanol was dropwise over 3 h, and then the reaction was heated for an additional 1 h. Water was added and the organic phase separated. Chloroform was added and the organic phase washed with water. The combined organic phases were dried with MgSO₄, filtered. The solvents and excess 1-chloro-3-bromopropane were removed under vacuum distillation to give a yield of 52% 3-bromopropyl methacrylate and 47% 3-chloropropyl methacrylate.

### EXAMPLE 6

### Commercial Process for Preparing (I)

Step (a):

4-THP-BA: 4-Hydroxybenzaldehyde (100.4 g, 0.82 mol), pyridinium p-toluenesulfonate (2.18 g, 0.011 eq) and p-toluenesulfonic acid (0.73 g, 0.005 eq) were dissolved in dichloromethane (900 cc, 17 eq). Thereafter 3,4-Dihydro-2H-pyran (110.37 g, 1.6 eq) was added dropwise, at room temperature, over 45 minutes. The reaction mixture was stirred for additional 1-2 hrs till complete. Most of dichloromethane was distilled at atmospheric pressure (˜720 g) and the concentrated reaction mixture was washed with 0.5M NaOH (1×133 g) and then with water (1×300 g). Turbidity was removed by simple gravitation filtration. Dichloromethane and other volatiles were removed by distillation, first at atmospheric pressure and then by applying vacuum at elevated temperature (up to 70° C.). Product is obtained as colorless oil (161 g, 95% yield).

Step (b):

Mono-nitrile: 4-THP-BA (204 g, 1.00 mol), 4-alkoxyphenylacetonitrile (1.35 eq), MeOH (1.18 L, 37.28 eq) and 1M NaOH (208 g, 0.2 eq) were mix together. Temperature was raised to reflux and reaction mixture was stirred for 6 hrs. After cooling to room temperature the product was collected and washed with methanol:water 9:1 (v/v, 2×365 g). Product is obtained as yellow powder (272 g, 81% yield).

Step (c):

Dinitrile: Mono-nitrile (85 g, 0.25 mol), sodium cynide (20.27 g, 1.63 eq), and water (126.6 g, 27.75 eq) were mixed at room temperature for 2 minutes to yield thick mixture. DMF (254.6 g, 13.76 g) was added and after 5 minutes MeOH (86.9 g, 10.72 eq) was added. Temperature in reaction mixture was raised to reflux and stirred for 4 hrs until reaction was complete. Water (241 g, 52.83 eq) were added to reaction mixture in 2 portions to aid complete precipitation and transfer the thick warm reaction mixture to Buchner funnel. Solvent was filtered away and the solid was washed with water (2×500 g) then with ethanol (2×253 g) and finally with t-butylmethylether (2×227 g). Product is obtained as white powder (87.45 g, 95% yield).

Step (d)

AS36: Di-nitrile (40 g, 0.11 mol), copper(II) acetate hydrate (0.7 g, 0.03 eq), piperidine (6.1 g, 0.65 eq), ethanolamine (3.03 gr 0.45 eq) and DMF (127.9 g, 15.87 eq) were mix together at room temperature to form blue solution. Air was bubbled in and reaction was stirred for 4 hrs until complete. Concentrated HCl (37%, 14.0 g) was introduced and the temperature was raised to 70° C. After 15 minutes, when THP protecting group was completely removed, water (160 g) were introduced dropwise over 40 minutes. Reaction mixture was cooled to room temperature and waited additional 40 minutes. Reaction mixture was filtered and the solid was washed with 1M HCl/0.1 M NaCl solution (3×150 g) than with water (2×200 g). A cis/trans product is obtained as yellow powder.

In order to enrich the mixture in trans isomer the dry mixture was heated in 1,2-dichlorobenzene (108 g) for ˜1 hr at 130° C. The solution was cooled to room temperature and after additional 2 hours the trans isomer was collected and washed with petroleum ether (2×35 g). 25.6 g of t-AS36 were obtained as yellow powder (84% yield).

Step (e)

NJ1: t-AS-36 (71.5 g, 0.26 mol) was dissolved in DMF (225 g, 11.9 eq) at 50° C. 40% NaOH solution was added (31.0 g, 1.2 eq). the intense red solution is stirred for 10 minutes and then 1,3-bromochloropropane was introduced in one portion. Three more portions of 20% NaOH (3×10.3 gr) were added till reaction was completed.

The pH in reaction was adjusted to 6 using 1M HCl (5.0 g). MeOH was introduced (220 g) and the reaction mixture was allowed to cool to room temperature. After additional 30 minutes product was collected and washed with water (2×320 g) and MeOH (2×220 g). The yellow powdered product (79.8 g, 87% yield), consists mainly in the chloride form.

Step (f)

MeAA: Acrylic acid (7.3 gr, 1.5 eq), potassium carbonate (5.6 g, 0.6 eq), sodium iodide (0.5 g, 0.05 eq) were mixed together in DMF (49.8 g, 10.07 eq) and the temperature was raised to 70° C. After evolving of CO₂ was ceased NJ1 (24.0 g, 0.068 mol) was introduced and temperature was raised to 90° C. After 4 hours, reaction was completed and MeOH (127 g) was added. Reaction mixture was cooled to room temperature over ˜1 hour and stirred for additional 2 hours to complete precipitation. MeAA was collected and washed with MeOH (1×70 g), water (2×134 g) and again with MeOH (1×70 g). Product is obtained as light yellow powder. If needed MeAA may be recrystalized from Methylethylketone:MeOH 3:4 w/w mixture. Typical yield is 80% after recrystallization.

1. A method of preparing a compound of formula (I) in the cis or trans geometry: 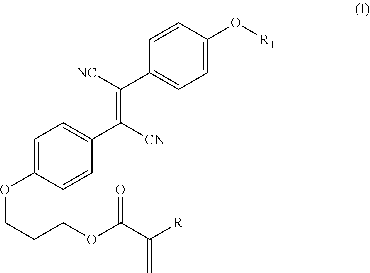
wherein R is selected from hydrogen and —CH₃ and R₁ is selected from —CH₃, —CH₂CH₃, —(CH₂)₂CH₃, and —(CH₂)₃CH₃, the method comprising:
a) reacting 4-hydroxybenzaldehyde with dihydropyran to yield the respective 4-tetrahydropyran-benzaldehyde of formula (A): 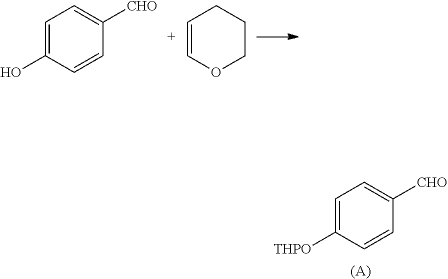
b) reacting the 4-tetrahydropyran-benzaldehyde (A) with 4-alkoxyphenylacetonitrile in the presence of a base to yield the mononitrile of formula (B): 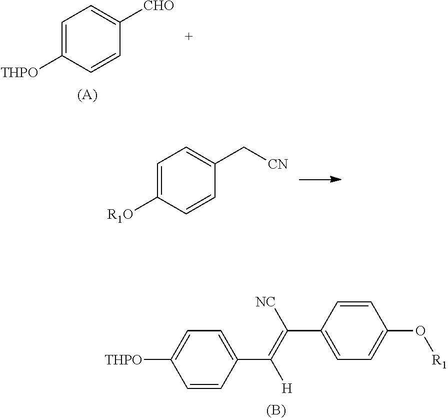
c) reacting the mononitrile of formula (B) with sodium cyanide in dimethylformamide to yield the dinitrile of formula (C): 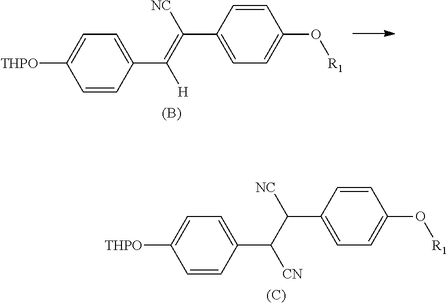
d) treating dinitrile of formula (C) with a mild base in the presence of copper(II) acetate hydrate to afford the compound of formula (D) in a mixture of cis and trans forms;
e) deprotecting (D) to yield the compound of formula (E) as a mixture of cis and trans forms;
f) enriching the trans isomer by heating the mixture of cis and trans forms of-(E) in the presence of dichlorobenzene to yield the predominantly trans-(F) compound: 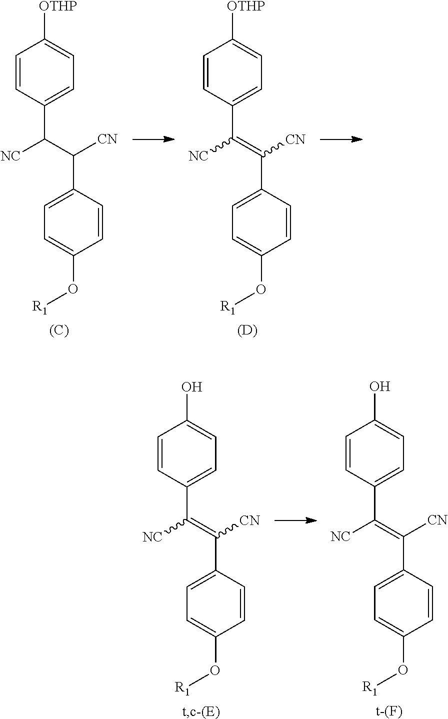
g) reacting the t-(F) with 1-chloro-3-bromopropyl in the presence of a base to yield the compound of formula (G): 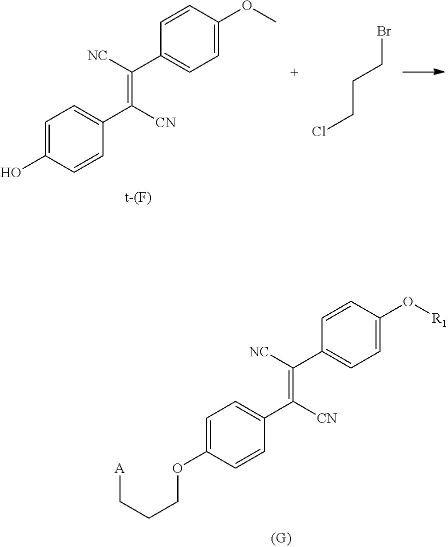
wherein A is selected from a chlorine and a bromine atom;
h) reacting the compound of formula (G) with acrylic (R═H)/methacrylic (R═CH₃) acid in the presence of a base to yield the compound of formula (I): 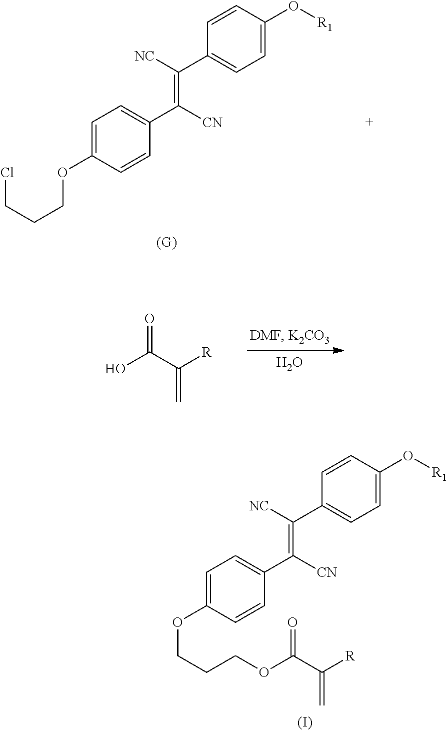
wherein R and R₁ are as defined above.

2. A method of preparing a compound of formula (I) in the cis or trans geometry: 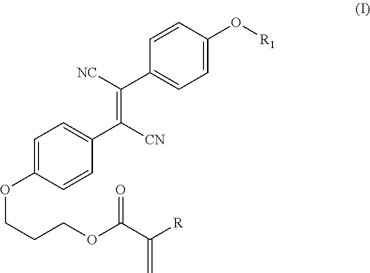
wherein R is selected from hydrogen and —CH₃ and R₁ is selected from —CH₃, —CH₂CH₃, —(CH₂)₂CH₃, and —(CH₂)₃CH₃, the method comprising:
a) homocoupling two 4-methoxyphenyl acetonitrile molecules in the presence of I₂, NaOMe/MeOH at a low temperature to afford 4,4′-dimethoxy-α,α-dicyanostilbene of formula (II); 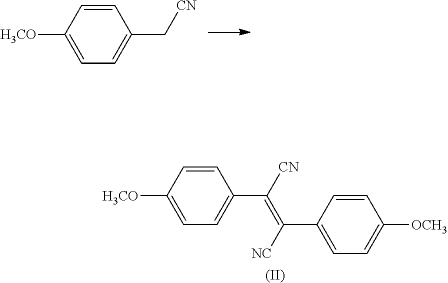
b) demethylating the compound of formula (II) by heating the compound (II) in the presence of AlCl₃/pyridine and DCB to yield 4,4′-dihydroxy-α,α-dicyanostilbene of formula (III); 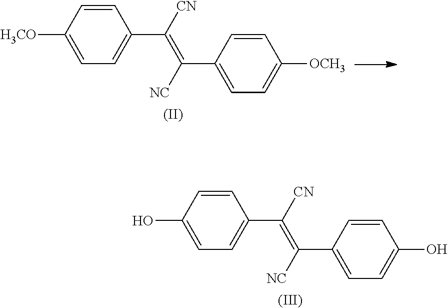
c) monoalkylating the compound of formula (III) with R₁X in the presence of a base in acetone to yield a 4-alkoxy-4′-hydroxy-α,α-dicyanostilbene of formula (IV), wherein X is a leaving group selected from Br, I and Cl; 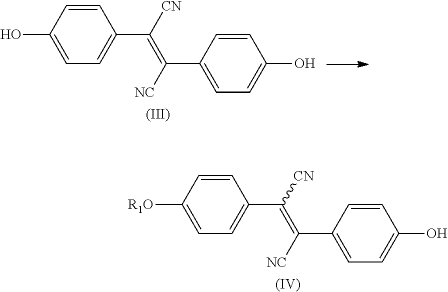
d) reacting the compound of formula (IV) with 3-bromopropyl-acrylate or methacrylate in the presence of base and acetone to yield a compound of formula (I) [chemical structure]
